(19) United States
(12) Patent Application Publication
(10) Pub. No.: US 2006/0292108 A1
(43) Pub. Date: Dec. 28, 2006

(54) Topical skin mask for oily and acneic skin

(76) Inventors: Ibrahim Hanna, Allentown, PA (US); Madiha Elaguizi, Allentown, PA (US); John Hanna, Allentown, PA (US); Haidy Hanna, Allentown, PA (US)

Correspondence Address: HANNA IBRAHIM;1St FLOOR, 443 NORTH 22nd STREET, ALLENTOWN, PA, 18104, US

(21) Appl. No.: 11/167,742

(22) Filed: Jun. 28, 2005

(51) Int. Cl. A61K 8/97

(52) U.S. Cl. 424/74

(57) ABSTRACT

A very efficient topical skin mask to reduce sebum, redness and inflammation of oily and acneic skin. The composition is produced by blending specific quantities of Zinc oxide, Kaolin, Lupini powder, Ascorbic acid and Sulfur, with Glycerin, Rose water and Distilled Water at room temperature and mixing with a high speed blender. The final product is a soft creamy mask to be applied to the surface of the skin.

### BACKGROUND OF THE INVENTION

The present invention relates to skin care preparations aiming to reduce the sebum as well as the redness and inflammation of oily and acneic skin.

Oiliness of the skin is caused by an abundant reservoir of oil residing in the sebaceous glands just below the surface of the skin, the sebum. It is secreted by the sebaceous gland found in the epidermis of the entire body except palms, soles and upper surfaces of the feet. These glands are found in their highest numbers on the head, particularly on the face and the scalp. Sebum secretion is under control of dihydrotestosterone (sexually related hormone). Sebum contains triglycerides, waxes, squalene and as it passes up the pilosebaceous duct, bacterial enzymes breakdown the triglycerides into free fatty acids.

The bacteria responsible for this are Corynebacterium acnes and staphylococcus aureus. Some of these free acids are known to potentiate obstruction of the pilosebaceous duct.

Excess sebum can contribute to the packing of horny skin cells at the opening of the sebaceous follicle on the surface of the skin, this causes a constriction in the follicular opening leading to a plug or comedone. There are open comedones and closed comedones:

A blackhead is an open comedo, with this type of obstruction, the sebaceous secretion can still escape to the surface and pilosebaceous duct rarely becomes inflamed.

A whitehead is a closed comedo, the pilosebaceous duct is obstructed completely and the sebaceous gland continues to excrete sebum. Bacteria present in the duct (Propionibacterium acnes) pour out lipases which breakdown triglycerides into free fatty acids promoting keratinization in the lamellae of the duct. Later, the gland ruptures behind the blockage and liberates sebum and free fatty acids into the surrounding epidermal and dermal tissues producing significant irritation, redness and tissue damage; sebum is the cause of acne redness.

Therefore prevention and treatment of acne is best achieved through reduction of sebum.

U.S. Pat. No.: 6,403,110 (SIDDIQUI)

Discloses a topical treatment for oily skin comprising an alcohol, pyridoxine hydrochloride and a zinc salt, to reduce sebum when applied to the surface of the skin.

U.S. Pat. No.: 4,372,296 (MOSTAFA)

Discloses a topical composition effective in the treatment of acne and skin disorders consisting of zinc sulfate, ascorbic acid, vitamin A and E in a pharmaceutical carrier such as water, alcohol, glycerol, oils or mixtures thereof.

U.S. Pat. No.: 2002037314 (FAXON)

Discloses an ascorbic acid based composition for the treatment of aging, photodamaged or inflammed skin. The composition includes ascorbic acid, non toxic zinc salt, sulfur containing anti-inflammatory compound and glucosamine, useful for treating rosacea and other inflammatory skin conditions.

U.S. Pat. No.: 2003180339 (KHAIAT)

Discloses the use of an enzymatically hydrolyzed cereal protein such as soy protein or wheat protein as a sebum regulating agent, preventing, controlling or inhibiting the oily shiny appearance and nature of acneic skin.

U.S. Pat. No.: 5,674,539 (ROBERT)

Discloses the use of Sulfur with antibiotics like Tetracyclin or Erythromycin in a topical medication for the treatment of skin pathology including acne.

Patent number: JP 60202813 (KEIISHI) et al

Discloses a skin composition for effective prevention and treatment of acne, comprising an aliphatic alcohol and sulfur as an active ingredient.

Patent number: JP 61225108 (YOSHIHARU) et al

Discloses a skin preparation comprising retinoic acid and sulfur for prevention and treatment of acne and prevention of dandruff.

Patent number: WO 9730719 (BINDICS FERENC, HU)

Discloses a preparation comprising a finely dispersed plant seed (Dried lentil, lens culinaris seeds) with boric acid and distilled water. The composition is advantageous for the treatment of seborrhea, purification of pores and fatty skin for prevention of acne.

Patent number: DE 3127639 (PAVEL, DE)

Discloses a cream for treatment of skin disorders containing as active ingredient Zinc oxide together with bismuth nitrate and red mercury sulfide.

### REFERENCES CITED

U.S Patent Documents

| | | | |
|---|---|---|---|
| 4372296 | FEBRUARY | 1983 | MOSTAFA |
| 5674539 | OCTOBER | 1997 | ROBERT |
| 2002037314 | MARCH | 2002 | FAXON |
| 6403110 | JUNE | 2002 | SIDDIQUI et al |
| 2003180339 | SEPTEMBER | 2003 | KHAIAT et al |
| DE 3127639 | AUGUST | 1982 | PAVEL |
| JP 60202813 | OCTOBER | 1985 | KEIICHI et al |
| JP 61225108 | OCTOBER | 1986 | YOSHIHARU et al |
| WO 9730719 | AUGUST | 1997 | FERENC, HU |
| RU 2185815 | JULY | 2002 | IORDANIDI |

### BRIEF SUMMARY OF THE INVENTION

Briefly, the invention is a topical mask for reducing sebum and inflammation (redness) on the surface of oily and acneic skin, comprising 12% zinc oxide, 10% kaolin, 10% Lupini beans powder, 8% ascorbic acid, 10% sulfur, 5% glycerin, 25% rose water and 20% distilled water.

Advantage of this Invention:

The presence of the seven active ingredients together provides a very good composition for reducing the sebum, redness and inflammation in the oily and acneic skin. They complemente and potentiate each other action giving best results.

### DETAILED DESCRIPTION OF THE INVENTION

The topical skin composition of this invention comprises a powder phase and a water phase processed together to produce a uniform mixture of the components: a soft creamy mask.

Referring to the first component of the topical skin mask of this invention, Zinc oxide: Topically applied Zinc has been used for decades for treatment of inflammatory acne.

In anti-acne medications, Zinc has a potent antiseptic effect (Dr. Provost) and more specifically, evidence suggests that Zinc have antibacterial and antifungal as well as mild anti-inflammatory actions (Drs. Rock, Wilkinson and Ebling).

In U.S Pat. No.: 4,372,296, a zinc salt and ascorbic acid are preferably present in an amount sufficient to provide a synergistic combination, effective as an antimicrobial agent against the microflora normally found in the pilosebaceous ducts (Corynebacterium acnes and staphylococcus aureus). At that concentration this combination will be also effective in the reduction of the sebum secretion rate. Zinc salt and ascorbic acid are mixed in a pharmaceutical carrier such as water, alcohol or glycerol so that not to inhibit their pharmacological activity.

Zinc oxide regulates the activity of sebaceous gland, is required for protein, DNA and RNA synthesis and collagen formation. It has mild astringent and antiseptic action, also it is an effective preservative.

Zinc oxide dissolves debris trapped deep within pores. Addition of sulfur further cleanses the skin and the mixture has a powerful antiseptic ability killing the bacteria. So Zinc and sulfur mask significantly reduce open (cystic) and closed (whiteheads) forms of acne.

Zinc is also believed to be useful in the treatment of acne, because of its ability to aid in wound healing, immune response, inflammation control, tissue regeneration and more effective utilization of vitamin A.

Certain studies have shown that Zinc produces results similar to tetracyclin in the treatment of superficial acne, but far more superior results with regard to deeper forms of acne (J. whitaker, Dr. whitaker guide to natural healing, 112/1995). Also Zinc is used with other ingredients for the treatment of rosacea.

The second component of the topical skin mask of this invention is Kaolin (alluminum silicate hydroxide). This clay is astringent, detoxifying, cleansing and stimulates lymph and blood circulation. Kaolin is an excellent mask for blackheads, whiteheads, acne spots and blemishes, because it absorbs excess oils and draws impurities from the skin. In patent number: RU 2185815 (IORDANIDI) a mask containing magnesium oxide, kaolin, talc and sulfur has bactericidal, anti-inflammatory and wound healing action.

The third component of the topical skin mask of this invention is Lupini powder. The lupin plant was first cultivated in Egypt two thousands years ago. The plant produces a white round seed that is very high in protein, carotene and alkaloids. Grinding these very hard beans gives a fine powder that can be used as a facial mask or scrub for face, elbows or for the whole body. The result is a wonderfully smooth, baby like skin with disappearance of dark spots.

The fourth component of the topical skin mask of this invention is Ascorbic acid (L-Ascorbic acid powder-powdered vitamin C). Its most well known effect is to stimulate immune function. Vitamin C is an antioxidant that protects tissues from tissue damage caused by free radicals.

Vitamin C also helps the body to repair damaged tissues, promoting wound healing as it is essential in the formation of collagen to protect against blood clotting and bruising.

Vitamin C also act as an antihistamine, so can be helpful for allergic problems. For its antioxidant effect and immune stimulating function, vitamin C has been found to protect against the harmful effects of pollution, prevents cancer and protects against infection.

Patented CSIRO technology provides transdermal absorption of 23% natural vitamin C. It is developped to improve the appearance of skin that shows dullness, coarse texture and to reduce fine lines and wrinkles associated with aging and sun exposure. It improves uneven discoloration and restores a smoother, softer and more youthful radiance. It decreases ultraviolet damage, lessens the severity of sunburns and stimulates collagen synthesis.

The fifth component of the topical skin mask of this invention is Sulfur. Sulfur is a pale yellow odorless brittle solid derived from volcanic ash in powder form. It is a minor constituent of fats, body fluids and skeletal minerals. It destroys bacteria causing acne. Sulfur is also called nature beauty mineral since it is found in keratin (protein) that strengthens skin and is needed for synthesis of collagen (Protein) that helps keep skin elastic and healthy. It desinfects the blood, helps the body to resist bacteria and protect the protoplasm of cells. Because of its ability to protect against harmful effects of radiation and pollution, sulfur slows the aging process.

For over two decades topical sulfur has been effectively used for treatment of rosacea papules and pustules (Drs. Ellis and Strawiski). Also Dr. Blom and Hornmark, Dr. Strauss used topical 10% sulfur preparation as a four weeks anti-rosacea treatment.

The desquamative agents are supposed to work by unblocking the follicles. Sulfur in various concentrations is still one of the most widely prescribed agents in this category as it helps breakdown of blackheads and whiteheads.

The sixth component of the topical skin mask of this invention is Glycerin, which acts as a humectant and moisturizer. It is derived from vegetable fatty acids, drawing vital moisture to the skin to insure flexibility and resilience. Glycerin penetrates the skin to help moisturize the deep layers. It softens the skin and is best for people with sensitive skin.

The seventh component of the topical skin mask of this invention is Rose Water. It stimulates and tones the skin. It is balancing for both dry and oily skin types as it controls and balances sebum production while having antibacterial properties. Its pH balancing and restoring effect helps tighten pores.

The Topical Skin Mask of this invention is applied to the surface of the skin once or twice daily according to the oiliness of the skin. It is left for 10 to 15 minutes then removed by gentle circular friction movements of the fingers (Gentle Scrub). Then the face is washed with warm water and dried.

Method

The topical skin mask of this invention is prepared as follows:

1—All of the powder phase ingredients: 12% Zinc oxide, 10% Kaolin, 10% Lupini seeds powder, 8% Ascorbic acid and 10% Sulfur powder, are put in a glass container and mixed at room temperature.

2—All of the water phase ingredients: 5% Glycerin, 25% Rose Water and 20% Distilled water are mixed together in a separate glass container at room temperature.

3—The water phase is gradually added to the powder phase while stirring.

4—The mixture is then vigorously blended at a high speed for 15 to 20 minutes to insure complete embodiment.

5—The final product is a soft creamy mask.

What we claim as our invention is:

1. A topical skin mask efficient to reduce sebum as well as redness and inflammation of oily and acneic skin. The composition consisting essentially of powder phase and water phase.

2. The powder phase of the topical skin mask of this invention consisting essentially of:
A. 12% Zinc oxide.
B. 10% Kaolin.
C. 10% Lupini powder.
D. 8% Ascorbic acid.
E. 10% Sulfur.

3. The water phase of the topical skin mask of this invention consisting essentially of:
A. 5% Glycerin.
B. 25% Rose water.
C. 20% Distilled water.
A method for preparing the topical skin mask consisting of powder phase (claim 2) including Zinc oxide, Kaolin, Lupini powder, Ascorbic acid, Sulfur and water phase (claim 3) including glycerin, rose water and distilled water:
The water phase (claim 3) is gradually added to the powder phase (claim 2) at room temperature while stirring. Mixing is continued for 15 to 20 minutes at high speed till the final composition becomes soft creamy mask.
